(19) United States
(12) Patent Application Publication
(10) Pub. No.: US 2024/0359015 A1
(43) Pub. Date: Oct. 31, 2024

(54) SYSTEMS AND METHODS FOR MODIFYING STIMULATION IN RESPONSE TO A CHANGE IN A SYMPTOM, THERAPEUTIC EFFECT, OR SIDE EFFECT

(71) Applicant: Boston Scientific Neuromodulation Corporation, Valencia, CA (US)

(72) Inventors: G. Karl Steinke, Valencia, CA (US); Lisa Denise Moore, Glendale, CA (US); Mahsa Malekmohammadi, Sherman Oaks, CA (US)

(21) Appl. No.: 18/645,034

(22) Filed: Apr. 24, 2024

Related U.S. Application Data
(60) Provisional application No. 63/462,905, filed on Apr. 28, 2023.

(51) Int. Cl. A61N 1/36

(52) U.S. Cl. CPC A61N 1/36139; A61N 1/36132

(57) ABSTRACT

A method for adapting stimulation by a stimulation system to a change in a symptom, therapeutic effect, or side effect includes detecting a change in a symptom of a disease or disorder or in a therapeutic effect or a side effect of stimulation by the stimulation system; in response to the detecting, determining, by the stimulation system, a modification of one or more stimulation parameter values to adapt to the change in the symptom, the therapeutic effect, or the side effect; and at least one of the following: 1) suggesting to the patient or to a clinician adoption of the modification and, upon receiving affirmation from the patient or the clinician, stimulating the patient according to the modification of the one or more stimulation parameter values, or 2) automatically stimulating the patient according to the modification of the one or more stimulation parameter values.

### CROSS-REFERENCE TO RELATED APPLICATIONS

This application claims the benefit under 35 U.S.C. § 119(e) of U.S. Provisional Patent Application Ser. No. 63/462,905, filed Apr. 28, 2023, which is incorporated herein by reference.

### FIELD

The present disclosure is directed to methods and systems for stimulation of a patient. The present disclosure is also directed to methods and systems for modifying stimulation in response to a change in a symptom, therapeutic effect, or side effect.

### BACKGROUND

Implantable electrical stimulation systems have proven therapeutic in a variety of diseases and disorders. For example, deep brain stimulation systems have been used as a therapeutic modality for the treatment of Parkinson's disease, essential tremor, and the like.

Stimulators have been developed to provide therapy for a variety of treatments. A stimulator can include an implantable pulse generator (IPG), one or more leads, and an array of stimulator electrodes on each lead. The stimulator electrodes are in contact with or near the nerves, muscles, or other tissue to be stimulated. The pulse generator in the IPG generates electrical pulses that are delivered by the electrodes to body tissue.

Implantable medical devices (IMDs), including IPGs, typically have the capability to communicate data with an external device, such as a clinician programmer or a remote control, via a radio-frequency telemetry link or other wireless communication method. The clinician programmer can program the operating parameters of the implanted medical device. The remote control can switch programs. Modern implantable devices also include the capability for bidirectional communication so that information can be transmitted to the clinician programmer or remote control from the implanted device.

### BRIEF SUMMARY

One aspect is a method for adapting stimulation by a stimulation system to a change in a symptom, therapeutic effect, or side effect, the stimulation system including a stimulation device implanted in a patient. The method includes detecting a change in a symptom of a disease or disorder or in a therapeutic effect or a side effect of stimulation by the stimulation system; in response to the detecting, determining, by the stimulation system, a modification of one or more stimulation parameter values to adapt to the change in the symptom, the therapeutic effect, or the side effect; and at least one of the following: 1) suggesting to the patient or to a clinician adoption of the modification and, upon receiving affirmation from the patient or the clinician, stimulating the patient according to the modification of the one or more stimulation parameter values, or 2) automatically stimulating the patient according to the modification of the one or more stimulation parameter values.

Another aspect is a stimulation system that includes a memory having instructions stored thereon and a processor configured for executing the instructions to perform actions including detecting a change in a symptom of a disease or disorder or in a therapeutic effect or a side effect of stimulation by the stimulation system; in response to the detecting, determining, by the stimulation system, a modification of one or more stimulation parameter values to adapt to the change in the symptom, the therapeutic effect, or the side effect; and at least one of the following: 1) suggesting to a patient or to a clinician adoption of the modification and, upon receiving affirmation from the patient or the clinician, stimulating the patient according to the modification of the one or more stimulation parameter values, or 2) automatically directing stimulation of the patient according to the modification of the one or more stimulation parameter values.

Yet another aspect is a non-transient computer readable medium having stored thereon instructions for performing actions for adapting stimulation by a stimulation system to a change in a symptom, therapeutic effect, or side effect, the actions including detecting a change in a symptom of a disease or disorder or in a therapeutic effect or a side effect of stimulation by the stimulation system; in response to the detecting, determining, by the stimulation system, a modification of one or more stimulation parameter values to adapt to the change in the symptom, the therapeutic effect, or the side effect; and at least one of the following: 1) suggesting to a patient or to a clinician adoption of the modification and, upon receiving affirmation from the patient or the clinician, stimulating the patient according to the modification of the one or more stimulation parameter values, or 2) automatically directing stimulation of the patient according to the modification of the one or more stimulation parameter values.

In at least some aspects, the symptom or side effect is dyskinesia. In at least some aspects, the symptom or side effect is freezing of gait.

In at least some aspects, detecting the change includes detecting the change using a sensor disposed on or within the patient. In at least some aspects, detecting the change includes detecting the change from information provided to the stimulation system by the patient, a caregiver, or a clinician.

In at least some aspects, the method or actions further include logging the change into a memory of the stimulation system. In at least some aspects, the logging the change further includes logging a time of the change into the memory of the stimulation system. In at least some aspects, the method or the actions further include logging whether the modification of the one or more stimulation parameter values is successful in addressing the change.

In at least some aspects, the method or the actions further include, in response to the detecting, notifying at least one of the patient or the clinician of the change. In at least some aspects, detecting the change includes detecting the change when the change includes an increase or decrease by a predetermined magnitude of at least one measured indicator. In at least some aspects, detecting the change includes detecting the change when the change includes an increase or decrease for at least a predetermined period of time of at least one measured indicator. In at least some aspects, the method or the actions further include determining whether the modification of the one or more stimulation parameter values increases or decreases the symptom or side effect.

Another aspect is a method for modifying stimulation by a stimulation system including a stimulation device implanted in a patient in response to an identified trend. The method includes identifying, by the stimulation system and over a period of time, a trend of adjustment of the stimulation in response to a trigger event; defining a schedule for the adjustment of the stimulation based on the identified trend; and automatically, or with affirmation by the patient or clinician, directing the adjustment of the stimulation according to the schedule.

A further aspect is a stimulation system that includes a memory having instructions stored thereon and a processor configured for executing the instructions to perform actions including identifying, by the stimulation system and over a period of time, a trend of adjustment of the stimulation in response to a trigger event; defining a schedule for the adjustment of the stimulation based on the identified trend; and automatically, or with affirmation by the patient or clinician, directing the adjustment of the stimulation according to the schedule.

Yet another aspect is a non-transient computer readable medium having stored thereon instructions for performing actions for modifying stimulation by a stimulation system, the actions including identifying, by the stimulation system and over a period of time, a trend of adjustment of the stimulation in response to a trigger event; defining a schedule for the adjustment of the stimulation based on the identified trend; and automatically, or with affirmation by the patient or clinician, directing the adjustment of the stimulation according to the schedule.

In at least some aspects, the trend includes the adjustment of the stimulation at a particular time of day. In at least some aspects, the trend includes the adjustment of the stimulation in response to administration of medication. In at least some aspects, the trend includes the adjustment of the stimulation in response to a determination that the patient is engaging in a specified activity.

In at least some aspects, the trend includes the adjustment of the stimulation in response to a change in a symptom of a disease or disorder or in a therapeutic effect or a side effect of stimulation by the stimulation system. In at least some aspects, the method or the actions further include detecting the change when the change includes an increase or decrease for at least a predetermined period of time of at least one measured indicator. In at least some aspects, the method or the actions further include detecting the change when the change includes an increase or decrease by a predetermined magnitude of at least one measured indicator. In at least some aspects, the method or the actions further include detecting the change using a sensor disposed on or within the patient.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes one or more leads that can be coupled to an IPG;

FIG. 2 is a block diagram of elements of an electrical stimulation system;

FIG. 3A is a schematic perspective view of a distal portion of one embodiment of an electrical stimulation lead with segmented electrodes;

FIG. 3B is a schematic perspective view of a distal portion of another embodiment of an electrical stimulation lead with segmented electrodes;

FIG. 3C is a schematic perspective view of a distal portion of a third embodiment of an electrical stimulation lead with segmented electrodes;

FIG. 3D is a schematic perspective view of a distal portion of a fourth embodiment of an electrical stimulation lead with segmented electrodes;

FIG. 3E is a schematic perspective view of a distal portion of a fifth embodiment of an electrical stimulation lead with segmented electrodes;

FIG. 4 is a flowchart of one embodiment of a method for adapting stimulation by a stimulation system to changes in a symptom, therapeutic effect, or side effect;

FIG. 5 is a flowchart of another embodiment of a method for adapting stimulation by a stimulation system to changes in a symptom, therapeutic effect, or side effect; and

FIG. 6 is a flowchart of one embodiment of a method of modifying stimulation in response to an identified trend.

### DETAILED DESCRIPTION

The present disclosure is directed to methods and systems for stimulation of a patient. The present disclosure is also directed to methods and systems for modifying stimulation in response to a change in a symptom, therapeutic effect, or side effect.

Implantable electrical stimulation systems and devices are used herein to exemplify the inventions, but it will be understood that these inventions can be utilized with other stimulation or modulation systems and devices, such as optical or electrical/optical stimulation or modulation systems. Examples of implantable electrical stimulation systems include, but are not limited to, a least one lead with one or more electrodes disposed along a distal end of the lead and one or more terminals disposed along the one or more proximal ends of the lead. Examples of electrical stimulation systems with leads are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 8,175,710; 8,224,450; 8,271,094; 8,295,944; 8,364,278; and 8,391,985; U.S. Patent Application Publications Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated by reference in their entireties. Examples of optical stimulation or modulation systems or electrical/optical stimulation systems, which include one or more optical emitters in addition to, or as an alternative to, electrodes, are found in U.S. Pat. Nos. 9,415,154; 10,335,607; 10,625,072; and 10,814,140 and U.S. Patent Application Publications Nos. 2013/0317572; 2013/0317573; 2017/0259078; 2017/0225007; 2018/0110971; 2018/0369606; 2018/0369607; 2019/0209849; 2019/0209834; 2020/0094047; 2020/0155584; 2020/0376262; 2021/0008388; 2021/0008389; 2021/0016111; and 2022/0072329, all of which are incorporated by reference in their entireties.

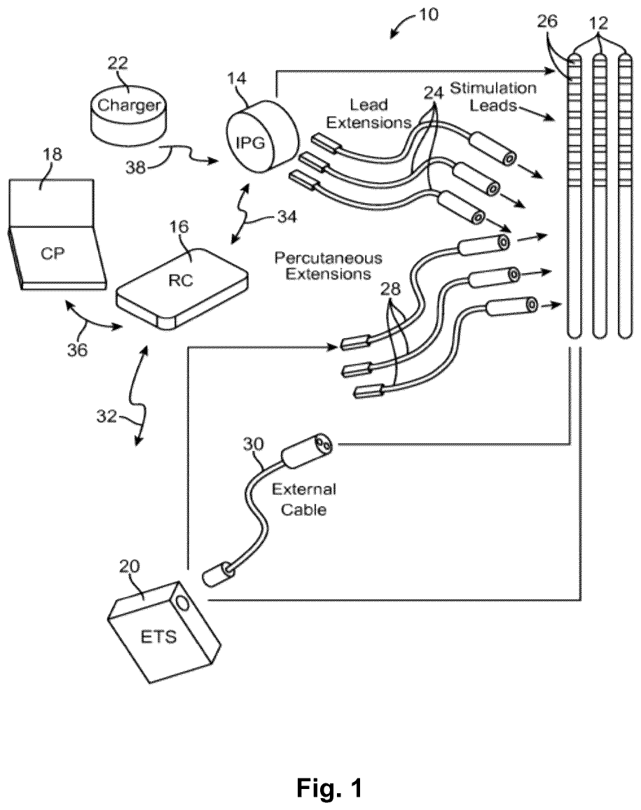

Turning to FIG. 1, one embodiment of an electrical stimulation system 10 includes one or more stimulation leads 12 and an implantable pulse generator (IPG) 14. The stimulation system 10 can also include one or more of an external remote control (RC) 16, a clinician's programmer (CP) 18, an external trial stimulator (ETS) 20, or an external charger 22. The IPG and ETS are examples of control modules for the electrical stimulation system.

The IPG 14 is physically connected, optionally via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameter values. The implantable pulse generator can be implanted into a patient's body, for example, below the patient's clavicle area or within the patient's abdominal cavity or at any other suitable site. The implantable pulse generator 14 can have multiple stimulation channels which may be independently programmable to control the magnitude of the current stimulus from each channel. In some embodiments, the implantable pulse generator 14 can have any suitable number of stimulation channels including, but not limited to, 4, 6, 8, 12, 16, 32, or more stimulation channels. The implantable pulse generator 14 can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameter values. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14, for example, to be turned on or off and to be programmed with different stimulation parameter sets. The IPG 14 may also be operated to modify the programmed stimulation parameter values to actively control the characteristics of the electrical stimulation energy output by the IPG 14. In at least some embodiments, the CP 18 (or RC 16 or other programming device) allows a user, such as a clinician, the ability to program stimulation parameter values for the IPG 14 and ETS 20 in the operating room and in follow-up sessions. Alternately, or additionally, in at least some embodiments, stimulation parameter values can be programed via wireless communications (e.g., Bluetooth) between the RC 16 (or other external device such as a hand-held electronic device like a mobile phone, tablet, or the like) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). In at least some embodiments, the stimulation parameter values provided by the CP 18 are also used to program the RC 16, so that the stimulation parameter values can be subsequently modified by operation of the RC 16 in a stand-alone mode (i.e., without the assistance of the CP 18). The CP 18 or RC 16 can be any suitable device including, but not limited to, a computer or other computing device, laptop, mobile device (for example, a mobile phone or tablet), or the like or any combination thereof. The CP 18 or RC 16 can include software applications for interacting with the IPG 14 or ETS 20 and for programming the IPG 14 or ETS 20.

Additional examples of the RC 16, CP 18, ETS 20, and external charger 22 can be found in the references cited herein as well as U.S. Pat. Nos. 6,895,280; 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, all of which are incorporated herein by reference in their entireties.

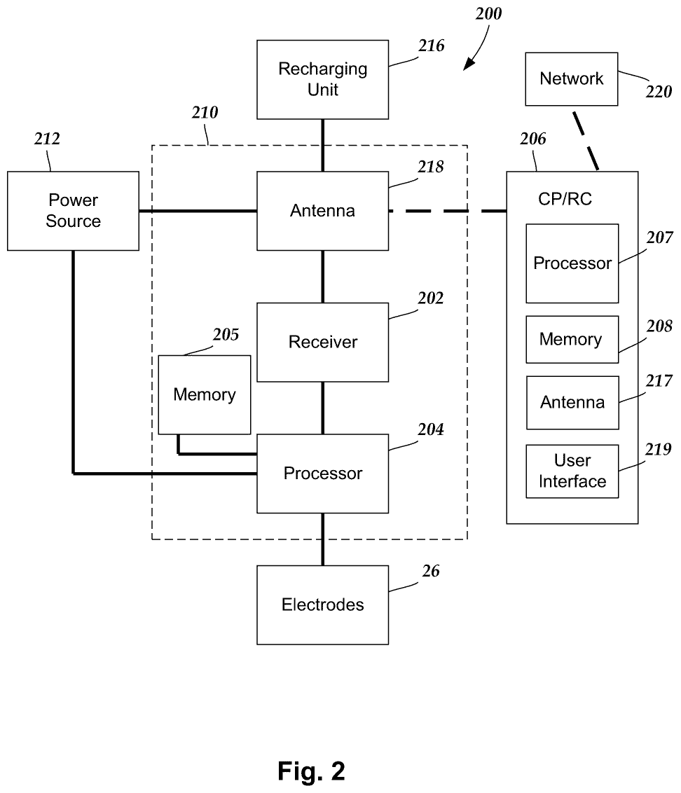

FIG. 2 is a schematic overview of one embodiment of components of an electrical stimulation system 200 including an electronic subassembly 210 disposed within an IPG 14 (FIG. 1). It will be understood that the electrical stimulation system can include more, fewer, or different components and can have a variety of different configurations including those configurations disclosed in the stimulator references cited herein.

The IPG 14 (FIG. 1) can include, for example, a power source 212, antenna 218, receiver 202, processor 204, and memory 205. Some of the components (for example, power source 212, antenna 218, receiver 202, processor 204, and memory 205) of the electrical stimulation system can be positioned on one or more circuit boards or similar carriers within a sealed housing of the IPG 14 (FIG. 1), if desired. Unless indicated otherwise, the term “processor” refers to both embodiments with a single processor and embodiments with multiple processors.

An external device, such as a CP or RC 206, can include a processor 207, memory 208, an antenna 217, and a user interface 219. The user interface 219 can include, but is not limited to, a display screen on which a digital user interface can be displayed and any suitable user input device, such as a keyboard, touchscreen, mouse, track ball, or the like or any combination thereof.

Any power source 212 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference in its entirety.

As another alternative, power can be supplied by an external power source through inductive coupling via the antenna 218 or a secondary antenna. The external power source can be in a device that is mounted on the skin of the user or in a unit that is provided near the user on a permanent or periodic basis.

If the power source 212 is a rechargeable battery, the battery may be recharged using the antenna 218, if desired. Power can be provided to the battery for recharging by inductively coupling the battery through the antenna to a recharging unit 216 external to the user. Examples of such arrangements can be found in the references identified above.

In one embodiment, electrical current is emitted by the electrodes 26 on the lead body to stimulate nerve fibers, muscle fibers, or other body tissues near the electrical stimulation system. A processor 204 is generally included to control the timing and electrical characteristics of the electrical stimulation system. For example, the processor 204 can, if desired, control one or more of the timing, frequency, amplitude, width, and waveform of the pulses. In addition, the processor 204 can select which electrodes can be used to provide stimulation, if desired. In some embodiments, the processor 204 may select which electrode(s) are cathodes and which electrode(s) are anodes. In some embodiments, the processor 204 may be used to identify which electrodes provide the most useful stimulation of the desired tissue. Instructions for the processor 204 can be stored on the memory 205. Instructions for the processor 207 can be stored on the memory 208.

Any processor 204 can be used for the IPG and can be as simple as an electronic device that, for example, produces pulses at a regular interval or the processor can be capable of receiving and interpreting instructions from the CP/RC 206 (such as CP 18 or RC 16 of FIG. 1) that, for example, allows modification of pulse characteristics. In the illustrated embodiment, the processor 204 is coupled to a receiver 202 which, in turn, is coupled to the antenna 218. This allows the processor 204 to receive instructions from an external source to, for example, direct the pulse characteristics and the selection of electrodes, if desired. Any suitable processor 207 can be used for the CP/RC 206.

Any suitable memory 205, 208 can be used including computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include, but are not limited to, RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a processor.

In one embodiment, the antenna 218 is capable of receiving signals (e.g., RF signals) from an antenna 217 of a CP/RC 206 (see, CP 18 or RC 16 of FIG. 1) which is programmed or otherwise operated by a user. The signals sent to the processor 204 via the antenna 218 and receiver 202 can be used to modify or otherwise direct the operation of the electrical stimulation system. For example, the signals may be used to modify the pulses of the electrical stimulation system such as modifying one or more of pulse width, pulse frequency, pulse waveform, and pulse amplitude. The signals may also direct the electrical stimulation system 200 to cease operation, to start operation, to start signal acquisition, to stop signal acquisition, to start charging the battery, or to stop charging the battery. In other embodiments, the stimulation system does not include an antenna 218 or receiver 202 and the processor 204 operates as programmed.

Optionally, the electrical stimulation system 200 may include a transmitter (not shown) coupled to the processor 204 and the antenna 218 for transmitting signals back to the CP/RC 206 or another unit capable of receiving the signals. For example, the electrical stimulation system 200 may transmit signals indicating whether the electrical stimulation system 200 is operating properly or not or indicating when the battery needs to be charged or the level of charge remaining in the battery. The processor 204 may also be capable of transmitting information about the pulse characteristics so that a user or clinician can determine or verify the characteristics.

Transmission of signals can occur using any suitable method, technique, or platform including, but not limited to, inductive transmission, radiofrequency transmission, Bluetooth™, Wi-Fi, cellular transmission, near field transmission, infrared transmission, or the like or any combination thereof. In addition, the IPG 14 can be wirelessly coupled to the RC 16 or CP 18 using any suitable arrangement include direct transmission or transmission through a network, such as a local area network, wide area network, the Internet, or the like or any combination thereof. The CP 18 or RC 16 may also be capable of coupling to, and sending data or other information to, a network 220, such as a local area network, wide area network, the Internet, or the like or any combination thereof.

At least some of the stimulation electrodes can take the form of segmented electrodes that extend only partially around the perimeter (for example, the circumference) of the lead. These segmented electrodes can be provided in sets of electrodes, with each set having electrodes circumferentially distributed about the lead at a particular longitudinal position.

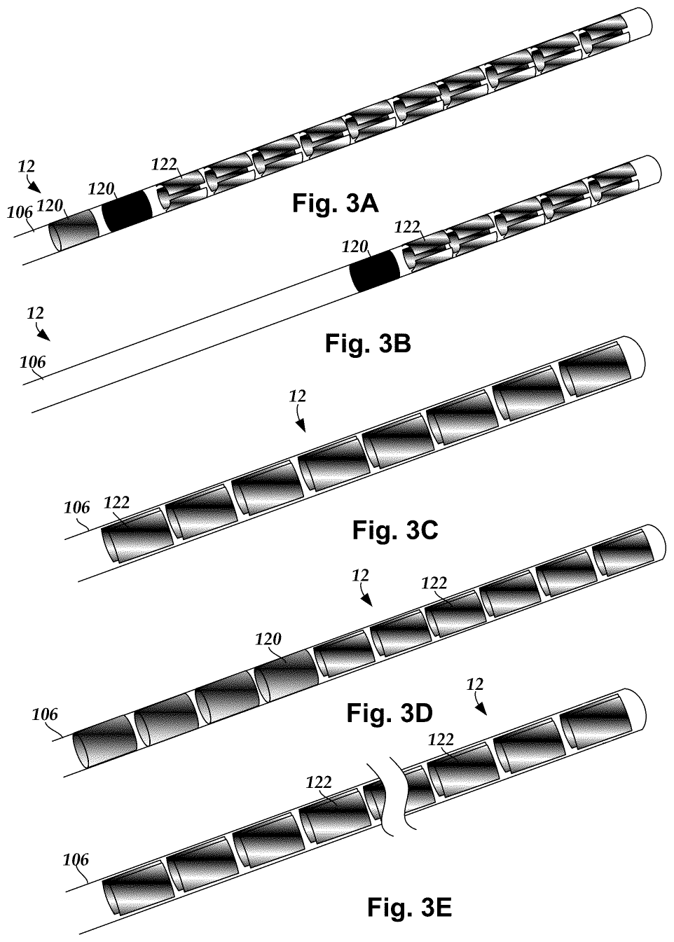

In FIGS. 3A, 3B, and 3D the electrodes are shown as including both ring electrodes 120 and segmented electrodes 122. In some embodiments, the electrodes are all segmented electrode 122, as illustrated in FIGS. 3C and 3E. The segmented electrodes 122 of FIG. 3A are in sets of three, where the three segmented electrodes of a particular set are electrically isolated from one another and are circumferentially offset along the lead 12. Any suitable number of segmented electrodes can be formed into a set including, for example, two, three, four, or more segmented electrodes. The lead 12 of FIG. 3A has thirty segmented electrodes 122 (ten sets of three electrodes each) and two ring electrodes 120 for a total of 32 electrodes.

Segmented electrodes can be used to direct stimulus current to one side, or even a portion of one side, of the lead. When segmented electrodes are used in conjunction with an implantable pulse generator that delivers current stimulus, current steering can be achieved to deliver the stimulus more precisely to a position around an axis of the lead (i.e., radial positioning around the axis of the lead). Segmented electrodes may provide for superior current steering than ring electrodes because target structures in deep brain stimulation are not typically symmetric about the axis of the distal electrode array. Instead, a target may be located on one side of a plane running through the axis of the lead. Through the use of a segmented electrode array, current steering can be performed not only along a length of the lead but also around a perimeter of the lead. This provides precise three-dimensional targeting and delivery of the current stimulus to neural target tissue, while potentially avoiding stimulation of other tissue.

FIG. 3A illustrates a 32-electrode lead 12 with a lead body 106 and two ring electrodes 120 proximal to thirty segmented electrodes 122 arranged in ten sets of three segmented electrodes each. In the illustrated embodiments, the ring electrodes 120 are proximal to the segmented electrodes 122. In other embodiments, the ring electrodes 120 can be proximal to, or distal to, or any combination thereof.

Any number of segmented electrodes 122 may be disposed on the lead body including, for example, one, two, three, four, five, six, seven, eight, nine, ten, eleven, twelve, thirteen, fourteen, fifteen, sixteen, twenty, twenty-four, twenty-eight, thirty, thirty-two, or more segmented electrodes 122. It will be understood that any number of segmented electrodes 122 may be disposed along the length of the lead body. A segmented electrode 122 typically extends only 75%, 67%, 60%, 50%, 40%, 33%, 25%, 20%, 17%, 15%, or less around the circumference of the lead.

The segmented electrodes 122 may be grouped into sets of segmented electrodes, where each set is disposed around a circumference of the lead 12 at a particular longitudinal portion of the lead 12. The lead 12 may have any number of segmented electrodes 122 in a given set of segmented electrodes. The lead 12 may have one, two, three, four, five, six, seven, eight, or more segmented electrodes 122 in a given set. The lead 12 may have any number of sets of segmented electrode including, but not limited to, one, two, three, four, five, six, eight, ten, twelve, fifteen, sixteen, twenty, or more sets. The segmented electrodes 122 may be uniform, or vary, in size and shape. In some embodiments, the segmented electrodes 122 are all of the same size, shape, diameter, width or area or any combination thereof. In some embodiments, the segmented electrodes 122 of each circumferential set (or even all segmented electrodes disposed on the lead 12) may be identical in size and shape.

Each set of segmented electrodes 122 may be disposed around the circumference of the lead body to form a substantially cylindrical shape around the lead body. The spacing between individual electrodes of a given set of the segmented electrodes may be the same, or different from, the spacing between individual electrodes of another set of segmented electrodes on the lead 12. In at least some embodiments, equal spaces, gaps, or cutouts are disposed between each segmented electrode 122 around the circumference of the lead body. In other embodiments, the spaces, gaps, or cutouts between the segmented electrodes 122 may differ in size or shape. In other embodiments, the spaces, gaps, or cutouts between segmented electrodes 122 may be uniform for a particular set of the segmented electrodes 122, or for all sets of the segmented electrodes 122. The sets of segmented electrodes 122 may be positioned in irregular or regular intervals along a length of the lead body.

The electrodes of the lead 12 are typically disposed in, or separated by, a non-conductive, biocompatible material of a lead body 106 including, for example, silicone, polyurethane, and the like or combinations thereof. The lead body 106 may be formed in the desired shape by any process including, for example, extruding, molding (including injection molding), casting, and the like. Electrodes and connecting wires can be disposed onto or within a lead body either prior to or subsequent to a molding or casting process. The non-conductive material typically extends from the distal end of the lead body 106 to the proximal end of the lead body 106.

FIG. 3B to 3E illustrate other embodiments of leads with segmented electrodes 122. FIG. 3B illustrates a sixteen electrode lead 12 having one ring electrode 120 that is proximal to five sets of three segmented electrodes 122 each. FIG. 3C illustrates a sixteen electrode lead 12 having eight sets of two segmented electrodes 122 each. As illustrated in FIG. 3C, an embodiment of a lead 12 does not necessarily include a ring electrode. FIG. 3D illustrates a sixteen electrode lead 12 having four ring electrodes 120 that are proximal to six sets of two segmented electrodes 122 each. FIG. 3E illustrates a thirty-two electrode lead 12 having sixteen sets of two segmented electrodes 122 each (for clarity of illustration, not all of the electrodes are shown). It will be recognized that any other electrode combination of ring electrodes, segmented electrodes, or both types of electrodes can be used.

When the lead 12 includes both ring electrodes 120 and segmented electrodes 122, the ring electrodes 120 and the segmented electrodes 122 may be arranged in any suitable configuration. For example, when the lead 12 includes two or more ring electrodes 120 and one or more sets of segmented electrodes 122, the ring electrodes 120 can flank the one or more sets of segmented electrodes 122. Alternately, the two or more ring electrodes 120 can be disposed proximal to the one or more sets of segmented electrodes 122 or the two or more ring electrodes 120 can be disposed distal to the one or more sets of segmented electrodes 122.

The electrodes 120, 122 may have any suitable longitudinal length including, but not limited to, 2, 3, 4, 4.5, 5, or 6 mm. The longitudinal spacing between adjacent electrodes 120, 122 may be any suitable amount including, but not limited to, 1, 2, or 3 mm, where the spacing is defined as the distance between the nearest edges of two adjacent electrodes. In some embodiments, the spacing is uniform between longitudinally adjacent electrodes along the length of the lead. In other embodiments, the spacing between longitudinally adjacent electrodes may be different or non-uniform along the length of the lead.

Examples of leads with segmented electrodes include U.S. Patent Application Publications Nos. 2010/0268298; 2011/0005069; 2011/0078900; 2011/0130803; 2011/0130816; 2011/0130817; 2011/0130818; 2011/0078900; 2011/0238129; 2011/0313500; 2012/0016378; 2012/0046710; 2012/0071949; 2012/0165911; 2012/0197375; 2012/0203316; 2012/0203320; 2012/0203321; 2013/0197602; 2013/0261684; 2013/0325091; 2013/0317587; 2014/0039587; 2014/0353001; 2014/0358209; 2014/0358210; 2015/0018915; 2015/0021817; 2015/0045864; 2015/0021817; 2015/0066120; 2013/0197424; 2015/0151113; 2014/0358207; and U.S. Pat. No. 8,483,237, all of which are incorporated herein by reference in their entireties. A lead may also include a tip electrode and examples of leads with tip electrodes include at least some of the previously cited references, as well as U.S. Patent Application Publications Nos. 2014/0296953 and 2014/0343647, all of which are incorporated herein by reference in their entireties. A lead with segmented electrodes may be a directional lead that can provide stimulation in a particular direction using the segmented electrodes.

Instead of, or in addition to, the electrodes 120, 122, a lead 12 can include one or more optical emitters (which can, for example, replace any of the electrodes 120, 122). Each optical emitter can be a light source (for example, a light emitting diode (LED), light emitting transistor (LET), laser diode, a vertical cavity side-emitting laser (VCSEL), an organic light emitting diode (OLED), an organic light emitting transistor (OLET), a lamp, or the like) or can be a light emission region of an optical waveguide (for example, a fiber optic, optical fiber, lens, or any other suitable conveyance of light) or the like. Examples of leads with optical emitters can be found at U.S. Pat. Nos. 9,415,154; 10,335,607; 10,625,072; and 10,814,140 and U.S. Patent Application Publications Nos. 2013/0317572; 2013/0317573; 2017/0259078; 2017/0225007; 2018/0110971; 2018/0369606; 2018/0369607; 2019/0209849; 2019/0209834; 2020/0094047; 2020/0155584; 2020/0376262; 2021/0008388; 2021/0008389; 2021/0016111; and 2022/0072329; all of which are incorporated herein by reference in their entireties.

The following discussion uses electrical stimulation systems with electrodes as examples. It will be understood that the optical stimulation or modulation systems or electrical/optical stimulation systems can be used in place of the example electrical stimulation systems. Thus, a stimulation system can include electrodes, optical emitters, or any combination thereof. It will be understood that any suitable type of stimulation can be used including, but not limited to, deep brain stimulation, spinal cord stimulation, vagal stimulation, peripheral nerve stimulation, or the like or any combination thereof.

In at addition, the following discussion uses Parkinson's disease, as an example of a disease or disorder for treatment, as well as dyskinesia and freezing of gait (FOG), as examples of symptoms or side effects. It will be understood that the methods and systems described herein can be used to treat other diseases, disorders, and symptoms and to address other side effects.

The sensing or monitoring of one or more symptoms, therapeutic effects, or side effects can be used to modify, adjust, or alter (or suggest modifications, adjustments, or alterations of) stimulation to enhance therapeutic effects or reduce side effects or symptoms. As an example, deep brain stimulation can be used to treat Parkinson's Disease (or other diseases and disorders) and provide therapeutic effects to reduce symptoms or side effects, such as dyskinesia or freezing of gait (FOG). Although some sets of stimulation parameter values can alleviate or prevent dyskinesia or FOG, other sets of stimulation parameter values may exacerbate dyskinesia or FOG while addressing other Parkinson's symptoms or providing other therapeutic effects. Stimulation parameter values may be selected as a compromise or combination between treatments for different symptoms.

In some instances, the magnitude or effect of a symptom, such as dyskinesia, or a therapeutic effect or side effect (or even the existence of a side effect) may increase or decrease for any of a variety of reasons. It may be desirable to modify, adjust, or alter stimulation in response to the change in the symptom, therapeutic effect, or side effect or any combination thereof. In addition, the presence or administration of medication, or changes in the dosage, dosage interval, or time of effect of the medication, may alter a patient's response to stimulation, which may include increasing or decreasing the magnitude of a symptom, therapeutic effect, or side effect or may include a side effect manifesting or becoming existent.

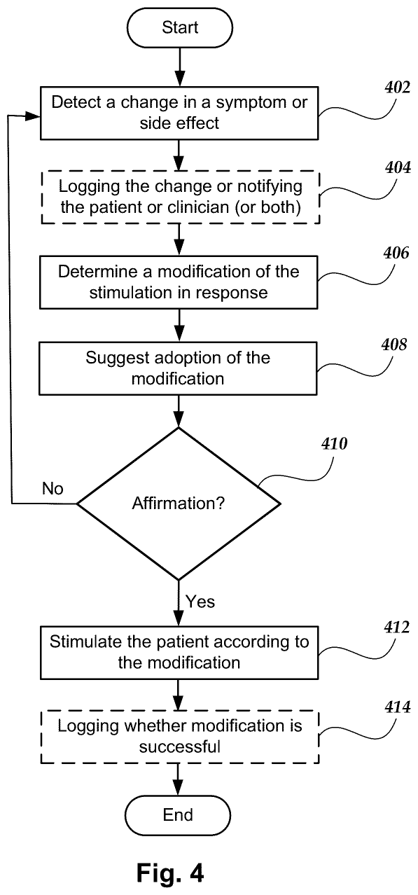

FIG. 4 illustrates one embodiment of a method for adapting stimulation by a stimulation system to changes in a symptom, therapeutic effect, or side effect. Examples of changes can include, but are not limited to, an increase or decrease in the magnitude of a symptom, therapeutic effect, or side effect; a change in the presentation of a symptom, therapeutic effect, or side effect; or manifestation or emergence of a symptom, therapeutic effect, or side effect. Examples of the symptom, therapeutic effect, or side effect include dyskinesia, freezing of gait, nausea, headache, dizziness, tingling or numbness in limbs, muscle contraction or twitching, fatigue or sleepiness, mood changes, anxiety, irritability, vision problems, cognitive or memory problems, cognitive impairment, paresthesia, affective disorder, akinesia, risk of falls, sleep or waking disturbance, apathy, depression, mania, punding or other compulsive disorders, dysarthria or other speech difficulties, changes in voice tone, hyperhidrosis, non-motor symptoms, or the like or any combination thereof.

In step 402, a change in a symptom of a disease or disorder or of a side effect of stimulation is detected or otherwise determined by the stimulation system. In at least some embodiments, a stimulation system can identify, monitor, or measure at least one symptom, therapeutic effect, or side effect (or an indicator of the symptom, therapeutic effect, or side effect). In at least some embodiments, a stimulation system can receive information from an external device to facilitate the identification, monitoring, or measurement of at least one symptom, therapeutic effect, or side effect (or an indicator of the symptom, therapeutic effect, or side effect). Any suitable method or device can be used for identifying, monitoring, or measuring including, for example, the use of one or more external or internal sensors; patient input (for example, information input by the patient into a remote control, which may be directed or non-directed); changes to the stimulation initiated by the patient, a clinician, or other individual; or the like or any combination thereof.

In at least some embodiments, a symptom of a disease/disorder, a therapeutic effect, or a side effect of stimulation is detected using a sensor disposed on or within the patient. Sensors, or other monitoring devices, can be used to identify dyskinesia, FOG, or other symptoms, therapeutic effects, or side effects. Sensors can be external, such as a smart watch, smart band, smart ring, smart phone or other smart device, shoe sensor, any other suitable wearable sensor, axially-located sensor, accelerometer, pressure sensor, temperature sensor, position sensor, auditory device. A sensor can be internal, such as implanted electrical, magnetic, or chemical sensors.

In at least some embodiments, a sensor can be selected to monitor or measure any suitable biosignal, chemical, or motion. As an example, a biosignal sensor can be used to monitor or measure local field potentials (LFP), evoked potential (EP), deep brain stimulation (DBS) local evoked potential (DLEP), evoked resonant neural activity (ERNA), ECG, EKG, EMG, electrocochleograph (ECOG), heart rate, blood pressure, electrical signals traversing the spinal cord or a nerve or group of nerves, any other suitable biological signals, or the like or any combination thereof. A chemical sensor can be used to monitor or measure, for example, any suitable biochemical(s). A motion sensor may be used to monitor, for example, tremor, gait, motion of particular body parts, or activities (for example, walking, running, sleeping, or the like), slowness of movement (e.g., bradykinesia), or any other suitable patient movement or motion, or the like or any combination thereof.

In at least some embodiments, an internal sensor can be part of the lead or control module. For example, the electrodes on a lead can also be used for recording a LFP, ERNA, ECG, EKG, EMG, electrocochleograph (ECOG), heart rate, ECG, or other electrical signals generated by or in the tissue of the patient.

In at least some embodiments, the sensitivity of the stimulation system to changes can be set or user selectable. For example, in order to suggest or initiate a modification of stimulation parameter value(s), the stimulation system can have a set, or user-selected, requirement that the change correspond to a threshold magnitude or score, or a threshold amount of change, for the symptom, therapeutic effect, or side effect or for an indicator of the symptom, therapeutic effect, or side effect). Additionally or alternatively, the stimulation system can have a set, or user-selected, requirement for a specific period of time (for example, 1, 5, 10, 15, or 30 minutes or 1, 2, 3, 4, or 6 or more hours) during which the determined change in the symptom, therapeutic effect, or side effect is present in order to suggest or initiate a modification of stimulation parameter value(s). In at least some embodiments, the stimulation system may also detect when the change or the symptom or side effect is no longer present and then revert to the early stimulation parameter value(s).

In at least some embodiments, the stimulation system can have a set, or user-selectable, period of time (for example, 1, 2, 3, 4, 6, or more hours) before another suggestion or initiation of a modification of stimulation parameter value(s) can occur. In at least some embodiments, this period of time may depend on the estimated or expected response rate of the symptom, therapeutic effect, or side effect to changes in stimulation. In at least some embodiments, the stimulation system can have a set, or user-selectable, period of time (for example, 1, 2, 3, 4, 6, or more hours) during which a transition to the modification is made such, as for example, ramping from the unmodified stimulation parameter value(s) to the modified stimulation parameter value(s); changing the duty cycle of the stimulation; or alternating between the modified stimulation parameter value(s) and the unmodified stimulation parameter value(s) (with, in at least some embodiments, increasing application of the modified stimulation parameter value(s)).

In at least some embodiments, the stimulation system can have a set, or user-selectable, limit on the modification of the stimulation parameter value(s). For example, the stimulation system may limit the change in amplitude, the change in the direction of stimulation (for example, rotation of the stimulation about the lead), the change in the movement of the stimulation up or down the lead (for example, change in the electrode selection up or down the lead), or the like or any combination thereof.

In at least some embodiments, there may be multiple thresholds or interconnected rules, requirements, or limits for any of these set, or user-selectable, parameters. As an example, the stimulation system may suggest or initiate a modification of stimulation parameter value(s) when a particular symptom or side effect increases by 5% and may limit another modification to occurring no sooner than five minutes later. The stimulation system in this example may have another rule that if the particular symptom or side effect increases by 10% then the five minute time limit is removed.

In optional step 404 (which can occur after any of steps 402, 406, or 408), the detected or determined change in the symptom, therapeutic effect, or side effect is logged into a memory of any suitable component of the stimulation system, such as, for example, the implantable pulse generator, remote control, clinician programmer, or any other suitable device, or to a memory external to the system. In at least in some embodiments, the stimulation system also logs the symptom, therapeutic effect, or side effect. Other information that can be logged includes, for example, the time of the change, whether medication was administered prior to the change, the time at which the medication was administered, medication dosage, medication dosage interval, a proposed modification of stimulation parameter values (see, step 406), the magnitude of the change, the current stimulation parameter values, or the like or any combination thereof.

Additionally or alternatively, in optional step 404, the stimulation system can notify the patient, a clinician, a caregiver, or any other suitable individual or any combination thereof of the change in the symptom, therapeutic effect, or side effect. For example, the patient or caregiver may be notified on the remote control or a smartphone that receives information from the system or sensor(s). The clinician may be notified by the clinician programmer or by the system sending a message to the clinician via email, text, phone call, or any other suitable communication method or any combination thereof. The notification may include any of the information that is described above with respect to logging and may include, for example, a predicted outcome of the modified stimulation, a time since a last notification or modification, or the like or any combination thereof.

In at least some embodiments, the patient, clinician, caregiver, or any other suitable individual or any combination thereof is notified on a periodic basis (for example, every 1, 2, 4, 6, or 12 hours or every day or week or any other suitable time period). In at least some embodiments, a notification after modification of the stimulation, as described below, to indicate or confirm the outcome of the modification.

In step 406, in response to detection of the change in the symptom, side effect or therapeutic effect, a modification of one or more stimulation parameter values of the stimulation is determined by the stimulation system to adapt to the change in the symptom, side effect, or therapeutic effect. In this manner, the stimulation system can provide for adaptive adjustment of the stimulation to the sensed symptom(s), therapeutic effect(s), or side effect(s). It will be understood that modification of one or more stimulation parameters can be performed by selecting a different stimulation program or altering an existing or current stimulation program.

In at least some embodiments, the stimulation system may utilize a set of preprogrammed rules to determine the modification. In at least some embodiments, the stimulation system may utilize recorded results (for example, recorded clinical effects) of previous stimulations to the patient or to other patients to determine the modification. Examples of such information can include neuroimaging and electrophysical information from the patient or from a population of patients or any combination thereof. Examples of methods for obtaining and recording clinical effects can be found at, for example, U.S. Pat. Nos. 9,227,074; 9,248,296; 9,358,398; 9,474,903; 10,071,249; 10,357,657; 10,369,364; 10,603,498; and 10,716,505; U.S. Patent Application Publications Nos. 2014/0243926; 2014/0276707; 2014/0277282; 2014/0277284; 2018/0264278; 2020/0376263; 2020/0398057; and 2021/0023374; U.S. patent application Ser. No. 18/075,835; and U.S. Provisional Patent Applications Ser. Nos. 63/425,149 and 63/432,628, all of which are incorporated herein by reference in their entireties. Any other suitable method for determination of the modification can be used including, but not limited to those in any of the references cited herein including U.S. Pat. Nos. 8,326,433; 8,675,945; 8,831,731; 8,849,632; 8.958,615; 10,265,528; and 10,603,498; U.S. Patent Application Publications Nos. 2009/0287272; 2009/0287273; 2012/0314924; 2013/0116744; 2014/0122379; 2015/0066111; and 2022/0339448, all of which are incorporated herein by reference in their entireties.

As an example, the stimulation system may determine a modification to the stimulation parameter value(s) that moves the stimulation (via, for example, a change in electrode selection or electrode fractionalization) relative to the lead or that reduces the stimulation (via, for example, reducing the amplitude, pulse width, or pulse rate or any combination thereof) when the symptom or side effect is determined to be worse. (Electrode fractionalization is the relative distribution of the stimulation current or voltage among the electrodes of the lead.) Modification to the electrode selection or electrode fractionalization may, for example, move the stimulation distally or proximally relative to the lead or narrow/increase the circumferential extent of the stimulation or the like or any combination thereof. The stimulation system may determine a modification to the stimulation program that increases the stimulation (via, for example, increasing the amplitude, pulse width, or pulse rate or any combination thereof) when the symptom or side effect is alleviated. The stimulation system may take into account the magnitude of the symptom or side effect in selection of the new stimulation parameter values or stimulation program.

In at least some embodiments, the stimulation system may also include modifications that have been previously input or selected by a clinician or other individual to address a change in a particular symptom, side effect, therapeutic effect, or other circumstance (for example, previously input stimulation programs). The stimulation system can adopt the previously input modification which may include a rule or rules for circumstances for implementation of that modification.

In at least some embodiments, the modification may include changing the stimulation along one portion of the lead while maintaining the stimulation along another portion of the lead. In at least some embodiments, the modification can include initiating stimulation when stimulation was previously not provided; initiating stimulation to a particular anatomical region that is not currently being stimulated; or halting or reducing stimulation to a particular anatomical region. In at least some embodiments, the modification may include providing supplemental stimulation to the stimulation currently provided.

In step 408, adoption of the modification is suggested to the patient, clinician, caregiver, or the like or any combination thereof. In at least some embodiments, when the stimulation system provides a suggestion to modify the stimulation program to the patient, clinician, caregiver, or the like or any combination thereof, the system can request confirmation that the modification should be adopted. In at least some embodiments, the patient or caregiver can affirm the modification using the remote control, a smartphone, or any other suitable device. In at least some embodiments, the clinician can affirm the modification using the clinician programmer or any other suitable device. In at least some embodiments, notification of the modification is sufficient to initiate the modification.

In at least some embodiments, multiple sets of modifications may be determined and may include assessments of risk, effectiveness, or the like or any combination thereof. The individual(s) queried for adoption of the modification may be asked to select between the sets of modifications.

In at least some embodiments in which the suggested modification has been previously adopted, the stimulation system may skip steps 408 and 410 and proceed directly to step 412. In at least some embodiments, the stimulation system can include one or more rules regarding a threshold for “closeness” of a suggested modification and a previously adopted modification that may allow the stimulation system to skip steps 408 and 410 and proceed directly to step 412.

In step 410, it is determined whether the patient or clinician (or both) affirm or accept the modification. If no, then the procedure returns to step 402 or ends. If yes, then in step 412, the patient is stimulated using the modification of the one or more stimulation parameter values.

In optional step 414, it is determined whether the modification is successful and the result of that determination is logged on the stimulation system or elsewhere. As an example, a determination is made whether the modification of the one or more stimulation parameter values increases or decreases the symptom or side effect to evaluate success or lack of success. The determination may be made using any suitable method or device including the methods and devices described above for determining a change in the symptom, therapeutic effect, or side effect. The system may also log the magnitude of any change in the symptom, side effect, or therapeutic effect or any change of an indicator of the symptom, side effect, or therapeutic effect. The system may also log any other suitable information including the information described above with respect to step 404. In at least some embodiments, a stimulation effect may take time to manifest. The log include observations over a period of time and may identify effects that occur over the period of time.

The procedure may then repeat the steps until stimulation is terminated or until the system is directed to halt the procedure.

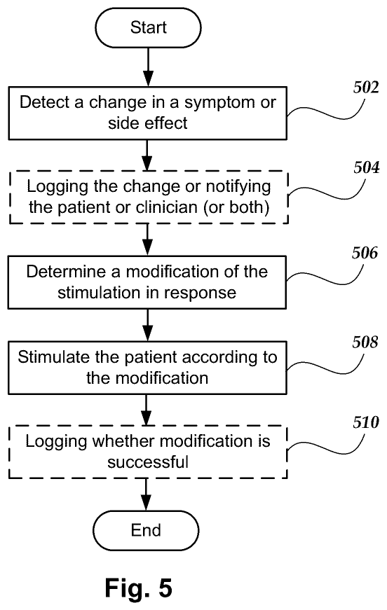

FIG. 5 illustrates another embodiment of a method for adapting stimulation by a stimulation system to changes in a symptom, therapeutic effect, or side effect. Steps 502, 504, 506, and 510 are the same as steps 402, 440, 406, and 414, respectively.

The method illustrated in FIG. 5 differs from the preceding method in that the modification of one or more of the stimulation parameter values is implemented automatically by the stimulation system, as illustrated in step 508. In at least some embodiments, the system allows, optionally without prompting, the patient, clinician, caregiver or the like or any combination thereof to reverse the modification. For example, when the patient is unsatisfied with the modified stimulation, the system can permit the patient to reverse the modification or otherwise change the stimulation.

In at least some embodiments, after the modification, the patient, clinician, caregiver, or the like or any combination thereof is informed of the change. In at least some embodiments, the informed individual(s) is/are requested to confirm the modification and, if not confirmed, the modification is reversed.

In at least some embodiments, the stimulation system records trends in stimulation program selection, modifications to stimulation parameter value(s), or the monitored level of at least one symptom, therapeutic effect, or side effect. As an example, when a symptom or side effect is detected or determined and a modification to one or more stimulation parameter values is initiated by the user, clinician, caregiver, or other individual or suggested or initiated by the stimulation system, the stimulation system can log the symptom or side effect (and, at least in some embodiments, the severity of the symptom or side effect) and the modification. Other information can be recorded, such as, for example, the time of the detection/determination of the symptom, therapeutic effect, or side effect, the time of the program change, the type of modifications made to the stimulation parameter value(s), the amount of change to the stimulation parameter value(s), administration of medication, time of the administration of medication, the results of stimulation using the modification (for example, decrease/increase in the symptom or side effect), or the like or any combination thereof.

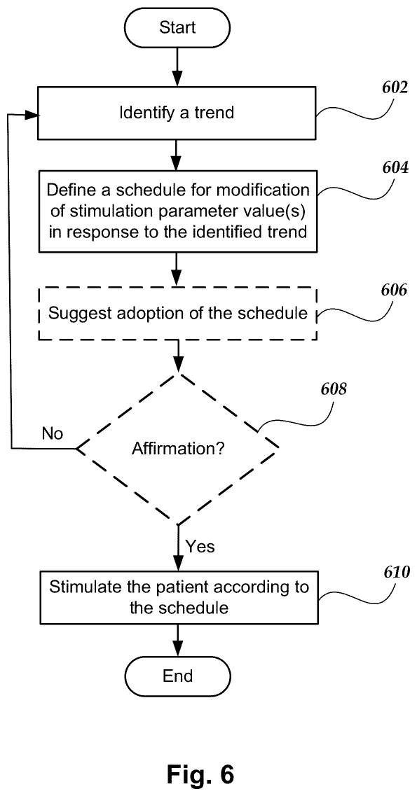

FIG. 6 illustrates one embodiment of a method of modifying stimulation in response to an identified trend. In step 602, the stimulation system, or an external system, can identify trends in the application or modification of specific stimulator parameter values generally or in response to a trigger event, such as, for example, changes in the symptom, therapeutic effect, or side effect; administration of medication; particular user activities or level of activity; particular time of day; or the like or any combination thereof. In at least some embodiments, the system can designate a modification as “successful” or “unsuccessful” (or any other equivalent words, numbers, or the like) based on the outcome of the stimulation, such as, for example, the decrease/increase of the symptom or side effect (or other symptoms or side effects or therapeutic effects or any combination thereof).

In step 604, in at least some embodiments, the stimulation system can define a schedule for suggesting, or automatically initiating, a modification of stimulation parameter value(s) based on the identified trend(s) (for example, in response to a trigger event). For example, when, over a course of time (for example, 1, 2, 5, or 10 days, 1, 2, or 4 weeks, 1, 2, 4, 6, or more months or any other suitable time period), the same or similar modifications to stimulation parameter value(s) are made or suggested under similar circumstances (for example, when the symptom or side effect increases, when medication is administered, or at the same time each day), the stimulation system can generate a schedule for modifying the stimulation parameter value(s) under specific conditions, such as detection/determination of the symptom or side effect, the administration of medication, specific times of the day, detection of specific activities, or the like or any combination thereof.

As an example, the stimulation system may identify a trend in the monitored level of at least one symptom, therapeutic effect, or side effect with administration of medication. The stimulation system may schedule the suggestion of, or automatically initiate, a different stimulation program to coincide with the administration of medication.

In optional step 606, adoption of the schedule is suggested to the patient, clinician, caregiver, or the like or any combination thereof. In at least some embodiments, when the stimulation system provides a suggested schedule to the patient, clinician, caregiver, or the like or any combination thereof, the system can request confirmation that the schedule should be adopted. In at least some embodiments, the patient or caregiver can affirm the schedule using the remote control, a smartphone, or any other suitable device. In at least some embodiments, the clinician can affirm the schedule using the clinician programmer or any other suitable device.

In optional step 608, it is determined whether the patient or clinician (or both) affirm or accept the schedule. If no, then the procedure returns to step 602 or ends. If yes, then in step 610, the patient is stimulated according to the schedule.

It will be understood that each block of the flowchart illustration, and combinations of blocks in the flowchart illustration and methods disclosed herein, can be implemented by computer program instructions. These program instructions may be provided to a processor to produce a machine or engine, such that the instructions, which execute on the processor, create means for implementing the actions specified in the flowchart block or blocks or engine disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computing device. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device. The computer program instructions can be stored locally or nonlocally (for example, in the Cloud).

The above specification and examples provide a description of the arrangement and use of the invention. Since many embodiments of the invention can be made without departing from the spirit and scope of the invention, the invention also resides in the claims hereinafter appended.

What is claimed as new and desired to be protected is:

1. A method for adapting stimulation by a stimulation system to a change in a symptom, therapeutic effect, or side effect, the stimulation system comprising a stimulation device implanted in a patient, the method comprising:
detecting a change in a symptom of a disease or disorder or in a therapeutic effect or a side effect of stimulation by the stimulation system;
in response to the detecting, determining, by the stimulation system, a modification of one or more stimulation parameter values to adapt to the change in the symptom, the therapeutic effect, or the side effect; and
at least one of the following:
16) suggesting to the patient or to a clinician adoption of the modification and, upon receiving affirmation from the patient or the clinician, stimulating the patient according to the modification of the one or more stimulation parameter values, or
2) automatically stimulating the patient according to the modification of the one or more stimulation parameter values.

2. The method of claim 1, wherein the symptom or side effect is dyskinesia.

3. The method of claim 1, wherein the symptom or side effect is freezing of gait.

4. The method of claim 1, wherein detecting the change comprises detecting the change using a sensor disposed on or within the patient.

5. The method of claim 1, wherein detecting the change comprises detecting the change from information provided to the stimulation system by the patient, a caregiver, or a clinician.

6. The method of claim 1, further comprising logging the change into a memory of the stimulation system.

7. The method of claim 6, wherein the logging the change further comprises logging a time of the change into the memory of the stimulation system.

8. The method of claim 6, further comprising logging whether the modification of the one or more stimulation parameter values is successful in addressing the change.

9. The method of claim 1, further comprising, in response to the detecting, notifying at least one of the patient or the clinician of the change.

10. The method of claim 1, wherein detecting the change comprises detecting the change when the change comprises an increase or decrease by a predetermined magnitude of at least one measured indicator.

11. The method of claim 1, wherein detecting the change comprises detecting the change when the change comprises an increase or decrease for at least a predetermined period of time of at least one measured indicator.

12. The method of claim 1, further comprising determining whether the modification of the one or more stimulation parameter values increases or decreases the symptom or side effect.

13. A method for modifying stimulation by a stimulation system comprising a stimulation device implanted in a patient in response to an identified trend, the method comprising:
identifying, by the stimulation system and over a period of time, a trend of adjustment of the stimulation in response to a trigger event;
defining a schedule for the adjustment of the stimulation based on the identified trend; and
automatically, or with affirmation by the patient or clinician, directing the adjustment of the stimulation according to the schedule.

14. The method of claim 13, wherein the trend comprises the adjustment of the stimulation at a particular time of day.

15. The method of claim 13, wherein the trend comprises the adjustment of the stimulation in response to administration of medication.

16. The method of claim 13, wherein the trend comprises the adjustment of the stimulation in response to a determination that the patient is engaging in a specified activity.

17. The method of claim 13, wherein the trend comprises the adjustment of the stimulation in response to a change in a symptom of a disease or disorder or in a therapeutic effect or a side effect of stimulation by the stimulation system.

18. The method of claim 17, further comprising detecting the change when the change comprises an increase or decrease for at least a predetermined period of time of at least one measured indicator.

19. The method of claim 17, further comprising detecting the change when the change comprises an increase or decrease by a predetermined magnitude of at least one measured indicator.

20. The method of claim 17, further comprising detecting the change using a sensor disposed on or within the patient.
